(19) United States
(12) Patent Application Publication
(10) Pub. No.: US 2009/0005577 A1
(43) Pub. Date: Jan. 1, 2009

(54) Method of producing 1-substituted 3-pyrrolates

(76) Inventor: Nikolai Kraiouchkine, Corpus Christi, TX (US)

Correspondence Address: Michael R. Ryes, 208 Poenisch Drive, Corpus Christi, TX, 78412, US

(21) Appl. No.: 12/215,569

(22) Filed: Jun. 28, 2008

Related U.S. Application Data
(60) Provisional application No. 60/947,391, filed on Jun. 29, 2007.

(51) Int. Cl. C07D 207/12

(52) U.S. Cl. 548/541

(57) ABSTRACT

The instant disclosure is directed to a method of producing crystals of a racemic mixture of a 1-substituted-3-pyrrolate. A method of producing crystals of racemic glycopyrrolate in high purity is also disclosed.

This Utility patent application claims benefit of the Provisional Application 60947391 filed on Jun. 29, 2007.

### BACKGROUND OF INVENTION

1. Field of the Invention

This invention relates to a process of producing 1-substituted 3-pyrrolates having excellent purity. More precisely, a method of producing a racemic mixture of glycopyrrolate in pure form, without distillation.

2. Background Art

1-substituted 3-pyrrolates are known in the art to be biologically active. These compounds are known in the art for providing beneficial results in a number of treatment plans. In particular, glycopyrrolate is known to have biological activity and is reported in the following references as being useful in treating medical conditions:

| | |
|---|---|
| U.S. Pat. No. 3,091,571 | antimalarial |
| U.S. Pat. No. 5,525,347 | treatment of performance anxiety; |
| U.S. Pat. No. 5,919,760 | treatment of acute and severe diarrhea; |
| U.S. Pat. No. 5,962,505 | treatment of hot flashes in humans; |
| U.S. Pat. No. 5,976,499 | use in a macroscopic sweat test for cystic |
| | fibrosis; |
| U.S. Pat. No. 6,063,808 | treating urinary incontinence; |
| U.S. Pat. No. 6,107,313 | dopamine receptor antagonist; |
| U.S. Pat. No. 6,204,285 | treating urinary incontinence; |
| U.S. Pat. No. 6,214,792 | treatment of acute and severe diarrhea; |
| U.S. Pat. No. 6,307,060 | use in medicaments; |
| U.S. Pat. No. 6,395,757 | treating hot flashes in humans; |
| U.S. Pat. No. 6,433,003 | treating hyperhidrosis; |
| U.S. Pat. No. 6,482,837 | antimuscarin compound, treatment of bladder |
| | diseases; |
| U.S. Pat. No. 6,585,958 | use in a medicinal aerosol formulation; |
| U.S. Pat. No. 6,613,795 | acetylcholine antagonist |
| U.S. Pat. No. 6,667,344 | bronchodilating composition; |
| U.S. Pat. No. 6,814,953 | bronchodilating composition; and |
| U.S. Pat. No. 7,060,289 | topical medicament. |

Many other examples of utility for glycopyrrolate are known. For more references, see Attachment A.

Preparation of 1-substituted 3-pyrrolidinols, and in particular 1-substituted 3-pyrrolates, is described in U.S. Pat. No. 2,956,062, which is incorporated herein by reference. While numerous approaches to produce 1-substituted 3-pyrrolates may exist, the byproducts produced require purification of intermediates via fractional distillation at reduced pressure in order to produce 1-substituted 3-pyrrolates of sufficient purity. This expensive and time consuming purification scheme may be responsible, at least in part, for the limited use of such compounds, especially in view of the plethora of uses such compounds enjoy. Accordingly, there is a need for a method to produce 1-substituted 3-pyrrolates in essentially pure form which does not require fractional distillation.

In addition, 1-substituted 3-pyrrolates are typically crystalline compounds with relatively high melting points. However, crystallization times of 1-substituted-3-pyrrolates from solution to solid is typically on the order of days, if not weeks. This facet may also contribute to the limited use of such compounds. Accordingly, a need exists for a method to produce 1-substituted 3-pyrrolates in essentially pure form, which includes crystallization of the salts in an economically viable time frame.

### SUMMARY OF INVENTION

In a first aspect of the present invention, a method of producing crystals of a racemic mixture of a 1-substituted-3-pyrrolate comprises the steps of:

a transesterification step comprising contacting, in a hydrocarbon solvent, an alkyl ester having the formula:

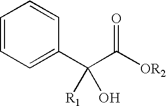

with a 1-alkyl substituted 3-pyrrolidinol having the formula:

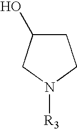

in the presence of a metallic sodium catalyst to produce a first reaction product comprising a free base of a 1-substituted-3-pyrrolidinol ester having the formula:

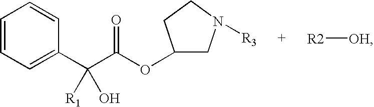

wherein R1 is a C1 to C10 alkyl, C5-C12 cycloalkyl, or a C6 C12 arylalkyl; R2 is a C1-C20 hydrocarbyl; and R3 is a C1-C10 alkyl or a C1-C10 cycloalkyl;

a diluting step comprising removing at least a portion of the hydrocarbon solvent and diluting the first reaction product with a mixture of a C1-C5 alcohol and ethyl acetate, to produce a second reaction mixture;

a quaternization step comprising cooling the second reaction mixture to a temperature below at least about 5° C. and contacting the second reaction mixture with a C1-C10 alkyl halide or a C5-C10 cycloalkyl halide, to produce a third reaction mixture comprising the 1-substituted-3-pyrrolate; and

a crystallization step comprising diluting the third reaction mixture with ethyl acetate to produce crystals comprising the 1-substituted-3-pyrrolate in a crystallization time of less than or equal to about 6 hours at 25° C., wherein the process does not include a fractional distillation step conducted under reduced pressure of the free base of the 1-substituted-3-pyrrolidinol ester.

In another aspect of the present invention, a process to produce crystals of a racemic mixture of glycopyrrolate comprises the steps of:

a transesterification step comprising contacting, in a hydrocarbon solvent, a methyl ester of cyclopentyl mandelic acid having the formula:

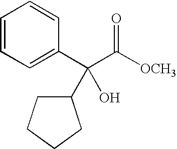

with 1-methyl 3-pyrrolidinol having the formula:

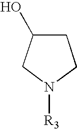

in the presence of a metallic sodium catalyst to produce a first reaction product comprising a free base of 3-[(cyclopentylhydroxyphenyl-acetyl)oxy]-1-methylpyrrolidine and MeOH;

a diluting step comprising removing at least a portion of the hydrocarbon solvent and diluting the first reaction product with a mixture of methanol and ethyl acetate, to produce a second reaction mixture;

a quaternization step comprising cooling the second reaction mixture to a temperature below at least about 5° C. and contacting the second reaction mixture with methyl bromide to produce a third reaction mixture comprising glycopyrrolate; and

a crystallization step comprising diluting the third reaction mixture with ethyl acetate to produce crystals comprising glycopyrrolate in a crystallization time of less than or equal to about 6 hours at 25° C., wherein the process does not include a fractional distillation step conducted under reduced pressure of the 3-[(cyclopentylhydroxyphenyl-acetyl)oxy]-1-methylpyrrolidine.

These and other features, aspects and advantages of the present invention will become better understood with reference to the following formulae, description and claims.

### DETAILED DESCRIPTION

The following detailed description is of the best currently contemplated modes of carrying out the invention. The description is not to be taken in a limiting sense, but is made merely for the purpose of illustrating the general principles of the invention, since the scope of the invention is best defined by the appended claims.

In the following description, numerous specific details are set forth to provide a thorough understanding of the present invention. However, it will be obvious to those skilled in the art that the present invention may be practiced without such specific details. In other instances, well-known methods and procedures have been shown in diagram form in order not to obscure the present invention in unnecessary detail. For the most part, details unnecessary to obtain a complete understanding of the present invention have been omitted in as much as such details are within the skills of persons of ordinary skill in the relevant art.

Broadly, the present invention generally provides a method to produce crystals of a 1-substituted-3-pyrrolidinol salt via transesterification of an ester with the pyrrolidinol in the presence of an alkali metal catalyst, followed by quaternization of the free base with an alkyl bromide to produce the 1-substituted-3-pyrrolate in a mixture of a C1-C5 alcohol and ethyl acetate. The salt is then crystallized from this mixture upon addition of ethyl acetate to produce the crystalline 1-substituted-3-pyrrolate. In an embodiment, the 1-substituted-3-pyrrolate is glycopyrrolate.

Glycopyrrolate is the common name for 3-[(cyclopentylhydroxyphenyl-acetyl)oxy]-1,1-dimethylpyrrolidinium bromide, also named 3-hydroxy-1,1-dimethylpyrrolidinium bromide α-cyclopentylmandelate. Glycopyrrolate is sold under the trade names AHR-504; Nodapton, Robanul; Robinul; Tarodyl; Tarodyn; and others. The empirical formula is C19H28BrNO3; Mol. Wt. 398.34 g/mol; has a reported melting point of 193.2-194.5° C., and is listed as an Anticholinergic by the Merck Index (monograph 4511, Merck Index 12th addition.) Glycopyrrolate has the following structure:

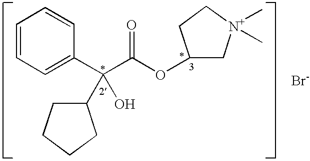

In the structural formula given above, the (*) atoms point out the asymmetric carbon atoms present in glycopyrrolate. One of the centers can be assigned to the acid moiety and relates to the position labeled 2′, the second chiral center is located in the cyclic ring system at the number 3 position. Since two asymmetric centers are present, two pairs of diastereoisomers are possible. Accordingly, four stereoisomers are possible, namely 3R, 2′R; 3S, 2′R; 3R, 2′S; and 3S, 2′S. It is to be understood that for purposes herein, the term glycopyrrolate refers to the racemic mixture of all four stereoisomers, indexed as CAS 596-51-0.

### DEFINITIONS

As used herein, the term “stereoisomers” refers to compounds which have identical chemical constitution, but differ with regard to the arrangement of the atoms or groups in space. In particular, “enantiomers” refer to two stereoisomers of a compound which are non-superimposable mirror images of one another. “Diastereomers”, on the other hand, refers to stereoisomers with two or more centers of dissymmetry and whose molecules are not mirror images of one another. With respect to the nomenclature of a chiral center, the terms “S” and “R” configuration are as defined by the IUPAC 1974 Recommendations for Section E., Fundamental Stereochemistry, Pure Appl. Chem., 45:13-30 (1976).

The notation numbering scheme for the Periodic Table Groups is used herein as set out in CHEMICAL AND ENGINEERING NEWS, 63(5), 27 (1985).

As used herein, concentrations may be expressed by a percentage of the material in the total composition. Unless otherwise stated, all percents express a weight percent (wt %), based on the amount of the material or component at issue in the total composition.

As used herein, the term hydrocarbyl refers to radicals comprising from 1 to 50 carbon atoms, and hydrogen atoms. Hydrocarbyl radicals may further include elements from Groups 13, 14, 15, 16, and 17 of the periodic table. Examples of hydrocarbyl groups include substituted and unsubstituted alkyl radicals, alkene radicals, alkyne radicals, aryl radicals, alicyclic radicals, and the like.

For brevity, upper and lower limitations on physical properties and process conditions may be expressed as ranges. However, it is to be understood that such ranges may comprise any combination of those upper and lower limits recited in any combination herein for a particular component, compound, composition, and/or process. While embodiments may be expressed as comprising a particular limitation, it is to be understood for use herein that such compositions may also consist of and/or consist essentially of the same limitations referred to herein as comprising a particular limitation.

In an embodiment, the 1-substituted-3-pyrrolate is produced by a process comprising a transesterification step, a diluting step, a quaternization step, and a crystallization step. These steps are outlined as follows:

transesterification Step

In the transesterification step, an alkyl ester of Formula I is contacted in a hydrocarbon solvent with a 1-alkyl substituted-3-pyrrolidinol of Formula II in the presence of a alkali metal catalyst at a temperature and for a period of time sufficient to produce a first reaction product comprising a 1-alkyl substituted-3-pyrrolidinol ester of Formula III as follows:

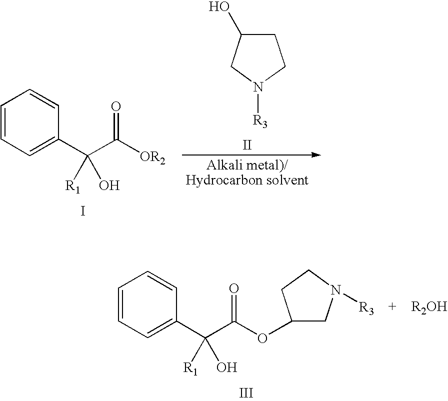

In an embodiment, R1 may be a C1 to C10 alkyl, C5-C12 cycloalkyl, or a C6-C12 arylalkyl. In a preferred embodiment, R1 is a C1 to C5 alkyl. In a still more preferred embodiment, R1 is a methyl group.

In an embodiment, R2 may be a C1-C20 hydrocarbyl. In another embodiment, R2 is a C1 to C5 alkyl. In still another embodiment, R2 is a methyl group.

In an embodiment, R3 may be a C1-C10 alkyl or a C1-C10 cycloalkyl. In another embodiment, R3 is a C1 to C5 alkyl. In still another embodiment, R3 is a methyl group.

The alkali metal catalyst is selected from Group 1 of the periodic table. Preferably the alkali metal catalyst is sodium (Na) or potassium (K), with Na being more preferred.

The hydrocarbon solvent may be a C5 to C12 hydrocarbon. In an embodiment, the hydrocarbon solvent is selected from the group consisting of hexanes, heptanes, octanes,

and any combination thereof. In an embodiment, the hydrocarbon solvent is essentially free from water. In still another embodiment, the hydrocarbon solvent is heptane.

In an embodiment, the alkyl ester of Formula I is contacted in the hydrocarbon solvent with the 1-alkyl substituted-3-pyrrolidinol of Formula II under reflux. In another embodiment, the transesterification step is conducted under reflux such that the alcohol produced is removed during the reaction. This may be accomplished in, for example, a “Dean-Stark” apparatus, or an equivalent thereof, as is understood in the art.

The transesterification step may further include washing the first reaction product by partitioning the reaction product against dilute acid (e.g., 10 wt % HCl aq.) to bring the 1-alkyl substituted-3-pyrrolidinol ester into the aqueous phase, with subsequent washing and/or via partition against hydrocarbon solvents and/or filtration to remove solids. The 1-alkyl substituted-3-pyrrolidinol ester may then be partitioned back into an organic phase by making the aqueous phase basic (e.g., via addition of potassium carbonate or another base known in the art) and then partitioning the basic aqueous phase against an organic solvent, more preferably against ethyl acetate. In an embodiment, the organic solvent may then be removed via rotary evaporation. In another embodiment, the final organic solution comprising the 1-alkyl substituted-3-pyrrolidinol ester in ethyl acetate may be utilized directly in the diluting step.

### The Diluting Step

In the diluting step, the 1-alkyl substituted-3-pyrrolidinol ester is dissolved in a mixture of a C1-C5 alcohol and ethyl acetate to produce a second reaction mixture. In an embodiment, the diluting step may include first removing the hydrocarbon solvent from the transesterification step and then adding the alcohol/ethyl acetate mixture to the first reaction product. In an alternative embodiment, the ethyl acetate is added at the conclusion of the washing steps as outlined above. Accordingly, the diluting step may include adding the C1-C5 alcohol to the ethyl acetate solution, without removal of the ethyl acetate resultant from the washing of Reaction Product 1.

In an embodiment, the mixture of the C1-C5 alcohol and ethyl acetate comprises methanol, ethanol, or a combination thereof. In an embodiment, the mixture of the C1-C8 alcohol and ethyl acetate comprises from about 0.1 to about 10 parts of the C1-C5 alcohol per part of ethyl acetate, by volume, preferably from about 0.5 to about 5 parts of the C1-C5 alcohol per part of ethyl acetate, more preferably from about 1 to about 2 parts of the C1-C5 alcohol per part of ethyl acetate, based on volume.

In another embodiment, the mixture of the C1-C5 alcohol and ethyl acetate comprises from about 0.1 to about 10 parts methanol per part of ethyl acetate, by volume, preferably from about 0.5 to about 5 parts of methanol per part of ethyl acetate, more preferably from about 1 to about 2 parts of methanol per part of ethyl acetate, based on volume (by volume).

In an embodiment, the mixture of C1-C5 alcohol and ethyl acetate comprises from about 0.1 to about 10 parts C1-C5 alcohol per part of ethyl acetate, by volume, preferably from about 0.16 to about 5 parts of C1-C5 alcohol per part of ethyl acetate, more preferably from about 0.125 to about 2 parts of C1-C5 alcohol per part of ethyl acetate by volume. In an embodiment, the mixture of C1-C5 alcohol and ethyl acetate comprises from about 1 part C1-C5 alcohol per about 6 to about 8 parts of ethyl acetate, by volume. In an embodiment, ethyl acetate may be replaced by methyl acetate, propyl acetate, isopropyl acetate, butyl acetate, sec-butyl acetate, t-butyl acetate, amyl acetate, iso-amyl acetate, or a combination thereof.

### Quateriization Step

The quaternization step comprises cooling of the second reaction mixture to a temperature below about 5° C. and contacting the second reaction mixture with an alkyl halide (R4-X) a temperature and for a period of time sufficient to produce a third reaction mixture comprising the 1-substituted-3-pyrrolate according to Formula IV. Formula IV

represents a salt (e.g., a methyl bromide salt) of the 1-alkyl substituted-3-pyrrolidinol ester, which is converted in this step from the free base to the salt as follows:

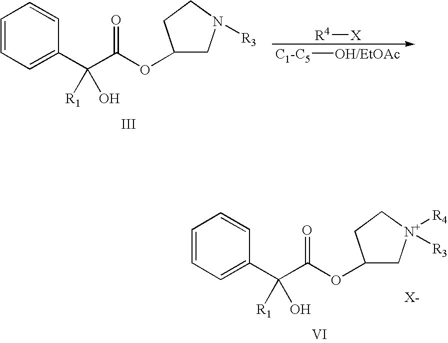

In an embodiment, R4 may include a C1-C10 alkyl or a C5-C10 cycloalkyl. In an embodiment, R4 is a C1-C10 alkyl, more preferably methyl. In an embodiment, the halide, “X” may include fluorine (F), chlorine (Cl), bromine (Br), and/or iodine (I), with Br and I being more preferred and with Br being still more preferred. In an embodiment, the alkyl halide is preferably methyl bromide, methyl iodide, or a combination thereof.

In an embodiment, the third reaction mixture is allowed to begin to warm to room temperature (e.g., about 25° C.) while the reaction is allowed to go to completion.

### The Crystallization Step

In the crystallization step, ethyl acetate is added to the reaction product of the quaternization step and the reaction mixture is allowed to warm to room temperature for a period of time sufficient to produce the crystals comprising the 1-substituted-3-pyrrolate according to Formula IV begin to form. The time in which these crystals form as determined from the end of the quaternization step to essentially the time in which the crystals cease to continue forming is referred to herein as the crystallization time. In an embodiment, the crystallization time is less than or equal to about 6 hours. In an embodiment, the crystallization time is less than or equal to about 5 hours, preferably less than or equal to about 4 hours, more preferably less than or equal to about 3 hours, more preferably less than or equal to about 2 hours, more preferably less than or equal to about 1 hour, more preferably less than or equal to about 0.5 hours, more preferably less than or equal to about 0.25 hours, with less than or equal to about 0.16 hours being more preferred. This is a major improvement over the art, wherein crystallization times were on the order of days and even weeks, depending of the solvent used during crystallization.

Once formed, the crystals comprising the substituted-3-pyrrolate according to Formula IV may be washed with 2-propanol, or another appropriate solvent. The crystals that then preferably dried e.g., via vacuum filtration, in a vacuum oven, and/or the like, and may be further characterized as required.

In an embodiment, the instant method is used to produce the racemic mixture of glycopyrrolate.

Glycopyrrolate is produced according to the instant method using the methyl ester of cyclopentyl mandelic acid (MeCMPA). MeCMPA useful herein is produced, preferably by reaction of cyclopentyl mandelic acid (CMPA) with dimethyl sulfate in the presence of a base at a temperature and for a sufficient time, typically about 5 to about 7 hours of reaction time at reflux. The methyl ester MeCMPA may also be prepared by Fischer esterification using methanol and a sulfuric acid catalyst (MeOH/H2SO4), as is common in the art.

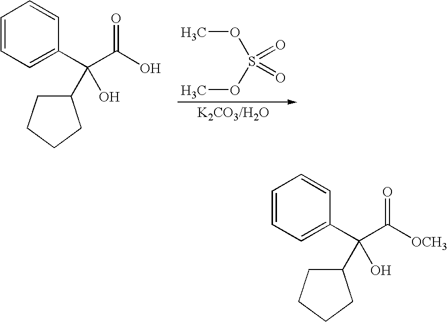

### Transesterification

In the transesterification step, the methyl ester of cyclopentyl mandelic acid (MeCPMA) is contacted (e.g., reacted under reflux) with N-methyl-3-pyrrolidinol in dry heptane with a sodium metal (Na (metal)) catalyst at a temperature and for a period of time sufficient to produce the free-base of glycopyrrolate (the “pyrrolidinol ester”), namely 3[(cyclopentylhydroxyphenyl-acetyl)oxy]-1-methylpyrrolidine and MeOH. Typical reaction conditions include reflux temperature for 2-3 hours of reaction time, with about 3 hours of time required for subsequent purification.

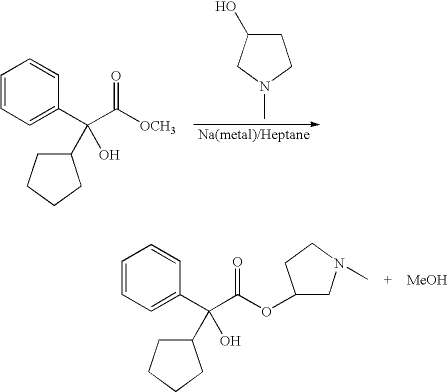

In an embodiment, the pyrrolidinol ester may then be extracted into an acidic aqueous phase via partition with dilute HCl in water. The aqueous phase is then filtered and may be washed with heptane. The aqueous phase is then made basic with dilute potassium carbonate and then partitioned against ethyl acetate. The free base of the pyrrolidinol ester is then extracted into the ethyl acetate. The pyrrolidinol ester in ethyl acetate is then washed with distilled water and subsequently dried using sodium sulfate. In an embodiment, the ethyl acetate solvent is then removed via rotary evaporation to produce the pyrrolidinol ester as an oil. In an alternative embodiment, the solution comprising the pyrrolidinol ester in ethyl acetate is continued to the diluting step.

### Diluting Step

In the diluting step, the pyrrolidinol ester is dissolved in a mixture of methanol (MeOH) and ethyl acetate (EtOAc.) In an embodiment, the mixture of methanol and ethyl acetate comprises from about 0.1 to about 10 parts methanol per part of ethyl acetate, by volume, preferably from about 0.16 to about 5 parts of methanol per part of ethyl acetate, more preferably from about 0.125 to about 2 parts of methanol per part of ethyl acetate by volume. In an embodiment, the mixture of methanol and ethyl acetate comprises from about 1 part methanol per about 6 to about 8 parts of ethyl acetate, by volume. In an embodiment, ethyl acetate may be replaced by methyl acetate, propyl acetate, isopropyl acetate, butyl acetate, sec-butyl acetate, t-butyl acetate, amyl acetate, iso-amyl acetate, or a combination thereof.

### Quaternization Step

In the quaternization step, the pyrrolidinol ester dissolved in the mixture of methanol and ethyl acetate is reacted with methyl bromide (MeBr) in a mixture of MeOH/EtOAc at ice bath temperature, which is less than or equal to about 5° C., with less than or equal to about 3° C. being more preferred, for a period of time sufficient to produce a racemic mixture of glycopyrrolate in MeOH/EtOAc.

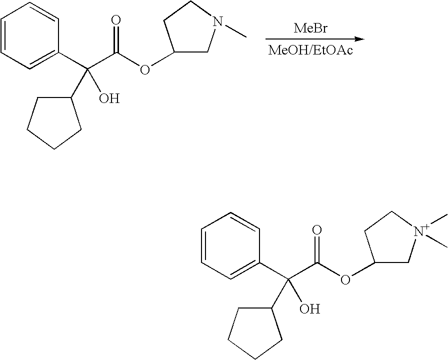

In an embodiment, the pyrrolidinol ester may be dissolved in a 1:1 mixture of MeOH/EtOAc, which is then cooled to a temperature of less than or equal to about 5° C. The reaction mixture is then contacted with methyl bromide for about 3 hours and the solution begins to warm to about 25° C. This reaction product is then added to an excess of ethyl acetate and crystals of the glycopyrrolate are allowed to form. The crystals form during a crystallization time of less than about 6 hours at 25° C. In an embodiment, the crystallization time is less than or equal to about 6 hours. In an embodiment, the crystallization time is less than or equal to about 5 hours, preferably less than or equal to about 4 hours, more preferably less than or equal to about 3 hours, more preferably less than or equal to about 2 hours, more preferably less than or equal to about 1 hour, more preferably less than or equal to about 0.5 hours, more preferably less than or equal to about 0.25 hours, with less than or equal to about 0.16 hours being more preferred. The crystalline glycopyrrolate is removed via vacuum filtration, and washed three times with 2-propanol. The glycopyrrolate crystals are then dried at 100° C. in a vacuum oven for about 0.5 to about 3 hours, with about 1 to about 2 hours being preferred. The melting point of the final dried crystals is then measured. The glycopyrrolate produced according to the instant method preferably has a melting point of greater than or equal to about 193.5° C., indicating an extremely pure material.

In an embodiment, if the melting point of the final dried crystals is less than 193.5° C., an optional recrystallization step may be conducted, wherein the crystals are redissolved in MeOH and once again crystallized using EtOAc and optionally a portion of methyl-t-butyl ether (MTBE) to produce glycopyrrolate crystals. These glycopyrrolate crystals are then washed with IPA, dried, and characterized as described above.

Although all three steps involve common knowledge, reactions, and purification procedures, the use of the MeOH/EtOAc mixture for quaternization and crystallization have unexpectedly allowed for a reduction in the overall process time, a reduction in the number of steps in the production, and a reduction in the amount of waste produced in the process. These improvements reduce both the expense of production, and the environmental impact of the instant process in terms of reducing the amount of waste produced.

In view of the prior art, the purification of the pyrrolidinol ester previously required distillation/fractionation under reduced pressure to remove residual oils. It has unexpectedly been discovered that this distillation/fractionation step can be removed from the procedure, as long as the quaternization and subsequent crystallization of the product is accomplished using the instant method.

The above described process results in a final product of glycopyrrolate having very high purity and requires only washing of the product with iso-propyl alcohol and optionally, a second recrystallization from methanol/ethyl acetate.

### EXAMPLES

### See Attachment B

It should be understood, of course, that the foregoing relates to preferred embodiments of the invention and that modifications may be made without departing from the spirit and scope of the invention as set forth in the following claims.

What is claimed:

1. A process to produce crystals of a racemic mixture of a 1-substituted-3-pyrrolate comprising the steps of:
a transesterification step comprising contacting, in a hydrocarbon solvent, an alkyl ester having the formula: 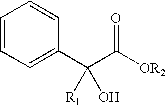
with a 1-alkyl substituted 3-pyrrolidinol having the formula: 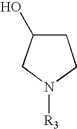
in the presence of a metallic sodium catalyst to produce a first reaction product comprising a free base of a 1-substituted-3-pyrrolidinol ester having the formula: 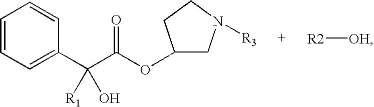
wherein R1 is a C1 to C10 alkyl, C5-C12 cycloalkyl, or a C6-C12 arylalkyl; R2 is a C1-C20 hydrocarbyl; and R3 is a C1-C10 alkyl or a C1-C10 cycloalkyl;
a diluting step comprising removing at least a portion of the hydrocarbon solvent and diluting the first reaction product with a mixture of a C1-C5 alcohol and ethyl acetate, to produce a second reaction mixture;
a quaternization step comprising cooling the second reaction mixture to a temperature below at least about 5° C. and contacting the second reaction mixture with a C1-C10 alkyl halide or a C5-C10 cycloalkyl halide, to produce a third reaction mixture comprising the 1-substituted-3-pyrrolate; and
a crystallization step comprising diluting the third reaction mixture with ethyl acetate to produce crystals comprising the 1-substituted-3-pyrrolate in a crystallization time of less than or equal to about 6 hours at 25° C., wherein the process does not include a fractional distillation step conducted under reduced pressure of the free base of the 1-substituted-3-pyrrolidinol ester.

2. The process of claim 1, wherein R1 is methyl, R2 is methyl, R3 is methyl, and the C1-C10 alkyl halide is methyl bromide.

3. The process of claim 1, wherein the C1-C5 alcohol is methanol, ethanol, or a combination thereof.

4. The process of claim 1, wherein the mixture of the C1-C5 alcohol and ethyl acetate comprises from about 0.1 to about 10 parts of the C1-C5 alcohol per part of ethyl acetate, by volume.

5. The process of claim 4, wherein the mixture of the C1-C5 alcohol and ethyl acetate comprises from about 0.1 to about 10 parts methanol per part of ethyl acetate, by volume.

6. The process of claim 4, wherein the mixture of the C1-C5 alcohol and ethyl acetate comprises from about 0.16 to about 5 parts methanol per part of ethyl acetate, by volume.

7. The process of claim 4, wherein the mixture of the C1-C5 alcohol and ethyl acetate comprises from about 1 part methanol to about 6 to about 8 parts of ethyl acetate, by volume.

8. The process of claim 1, wherein the crystallization time is less than or equal to about 0.25 hours at 25° C.

9. The process of claim 1, wherein the hydrocarbon solvent is hexane, heptane, octane, or a combination thereof.

10. A process to produce crystals of a racemic mixture of glycopyrrolate comprising the steps of:
a transesterification step comprising contacting, in a hydrocarbon solvent, a methyl ester of cyclopentyl mandelic acid having the formula: 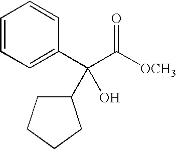
with a 1-methyl 3-pyrrolidinol having the formula: 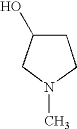
in the presence of a metallic sodium catalyst to produce a first reaction product comprising a free base of 3-[(cyclopentylhydroxyphenyl-acetyl)oxy]-1-methylpyrrolidine and MeOH;
a diluting step comprising removing at least a portion of the hydrocarbon solvent and diluting the first reaction product with a mixture of methanol and ethyl acetate, to produce a second reaction mixture;
a quaternization step comprising cooling the second reaction mixture to a temperature below at least about 5° C. and contacting the second reaction mixture with methyl bromide to produce a third reaction mixture comprising glycopyrrolate; and
a crystallization step comprising diluting the third reaction mixture with ethyl acetate to produce crystals comprising glycopyrrolate in a crystallization time of less than or equal to about 6 hours at 25° C., wherein the process does not include a fractional distillation step conducted under reduced pressure of the 3-[(cyclopentylhydroxyphenyl-acetyl)oxy]-1-methylpyrrolidine.

11. The process of claim 10, wherein the mixture of methanol and ethyl acetate comprises from about 0.1 to about 10 parts methanol per part of ethyl acetate, by volume.

12. The process of claim 10, wherein the mixture of methanol and ethyl acetate comprises from about 0.16 to about 5 parts methanol per part of ethyl acetate, by volume.

13. The process of claim 10, wherein the mixture of methanol and ethyl acetate comprises from about 1 part methanol to about 6 to about 8 parts of ethyl acetate, by volume.

14. The process of claim 10, wherein the crystallization time is less than or equal to about 0.25 hours at 25° C.

15. The process of claim 10, wherein the glycopyrrolate crystals have a melting point of greater than or equal to about 193.5° C.

16. A process to produce crystals of a racemic mixture of a 1-substituted-3-pyrrolate having the steps of a transesterification step comprising contacting, in a hydrocarbon solvent, an alkyl ester having the formula: 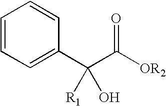
with a 1-alkyl substituted 3-pyrrolidinol having the formula: 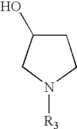
in the presence of a metallic sodium catalyst to produce a first reaction product comprising a free base of a 1-substituted-3-pyrrolidinol ester having the formula: 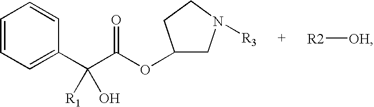
wherein R1 is a C1 to C10 alkyl, C5-C12 cycloalkyl, or a C6-C12 arylalkyl; R2 is a C1-C20 hydrocarbyl; and R3 is a C1-C10 alkyl or a C1-C10 cycloalkyl;
the improvement comprising a diluting step comprising removing at least a portion of the hydrocarbon solvent and diluting the first reaction product with a mixture of a C1-C5 alcohol and ethyl acetate, to produce a second reaction mixture;
a quaternization step comprising cooling the second reaction mixture to a temperature below at least about 5° C. and contacting the second reaction mixture with a C1-C10 alkyl halide or a C5-C10 cycloalkyl halide, to produce a third reaction mixture comprising the 1-substituted-3-pyrrolate; and
a crystallization step comprising diluting the third reaction mixture with ethyl acetate to produce crystals comprising the 1-substituted-3-pyrrolate in a crystallization time of less than or equal to about 6 hours at 25° C., wherein the process does not include a fractional distillation step conducted under reduced pressure of the free base of the 1-substituted-3-pyrrolidinol ester.

17. A process to produce crystals of a racemic mixture of glycopyrrolate having the steps of:
a transesterification step comprising contacting, in a hydrocarbon solvent, a methyl ester of cyclopentyl mandelic acid having the formula: 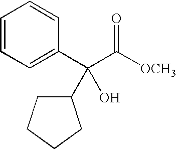
with a 1-methyl 3-pyrrolidinol having the formula: 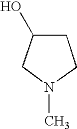
in the presence of a metallic sodium catalyst to produce a first reaction product comprising a free base of 3-[(cyclopentylhydroxyphenyl-acetyl)oxy]-1-methylpyrrolidine and MeOH;
the improvement comprising a diluting step comprising removing at least a portion of the hydrocarbon solvent and diluting the first reaction product with a mixture of methanol and ethyl acetate, to produce a second reaction mixture;
a quaternization step comprising cooling the second reaction mixture to a temperature below at least about 5° C. and contacting the second reaction mixture with methyl bromide to produce a third reaction mixture comprising glycopyrrolate; and
a crystallization step comprising diluting the third reaction mixture with ethyl acetate to produce crystals comprising glycopyrrolate in a crystallization time of less than or equal to about 6 hours at 25° C., wherein the process does not include a fractional distillation step conducted under reduced pressure of the 3-[(cyclopentylhydroxyphenyl-acetyl)oxy]-1-methylpyrrolidine.
